(19) United States
(12) Patent Application Publication
(10) Pub. No.: US 2025/0072870 A1
(43) Pub. Date: Mar. 6, 2025

(54) Adaptive Acoustic Imaging with Differentiable Beamforming

(71) Applicant: The Board of Trustees of the Leland Stanford Junior University, Stanford, CA (US)

(72) Inventors: Dongwoon Hyun, Sunnyvale, CA (US); Walter A. Simson, IV, Menlo Park, CA (US); Louise Leyi Zhuang, Stanford, CA (US); Jeremy J. Dahl, Palo Alto, CA (US)

(21) Appl. No.: 18/821,626

(22) Filed: Aug. 30, 2024

Related U.S. Application Data
(60) Provisional application No. 63/535,555, filed on Aug. 30, 2023.

(51) Int. Cl. A61B 8/08; G01S 7/52

(52) U.S. Cl. CPC A61B 8/483; A61B 8/5207; G01S 7/52098

(57) ABSTRACT

A method of ultrasound imaging includes a) performing a pulse-echo acquisition to produce channel signals using ultrasound transducers; b) processing transmitted and received channel signals to produce a dataset of a set of transmit elements and a set of receive elements; c) calculating an estimate of a critical imaging parameter; d) performing beamforming using the dataset and the estimate of the critical imaging parameter; e) calculating a desired loss function minimized with respect to the critical imaging parameter; f) differentiating the calculated loss function with respect to the critical imaging parameter by backpropagation; g) updating the estimate of the critical imaging parameter; h) repeating steps (d)-(g) until a convergence condition is satisfied; and i) generating an enhanced ultrasound image using the estimate of the critical imaging parameter.

### CROSS-REFERENCE TO RELATED APPLICATIONS

This application claims priority from U.S. Provisional Patent Application 63/535,555 filed Aug. 30, 2023, which is incorporated herein by reference.

### STATEMENT OF FEDERALLY SPONSORED RESEARCH

This invention was made with Government support under contracts EB032230 and EB027100 awarded by the National Institutes of Health, and under contract 3146755 awarded by the National Science Foundation. The Government has certain rights in the invention.

### FIELD OF THE INVENTION

The present invention relates generally to ultrasound imaging. More specifically, it relates to techniques for estimation of imaging parameters in ultrasound imaging.

### BACKGROUND OF THE INVENTION

Ultrasound imaging depends on critical imaging parameters that depend on physical quantities in the imaging system (including but not limited to position, material sound speed, material attenuation, material scatter density, material non-linearity, material motion, material deformation) that have been found empirically and are often statically set for a given imaging device. This configuration can degrade image quality in situations where these a priori assumptions break down.

### SUMMARY OF THE INVENTION

In one aspect, the present invention provides a differentiable formulation of the beamforming pipeline to optimize for critical imaging parameters which can improve image quality in ultrasound imaging, and simultaneously aid in diagnosis via the quantitative estimation of imaging system quantities. This differentiable beamformer has multiple use cases. For example, we show in test cases for sound speed estimation, element position estimation for flexible arrays, probe position estimation for freehand 3D ultrasound, and tissue deformation estimation for vector flow imaging or deformable registration, that this technology can aid in generating accurate and useful imaging quantitative parameter estimates.

This ultrasound imaging paradigm jointly achieves critical imaging parameter and image quality enhancement via differentiable beamforming. We formulate image reconstruction as a differentiable function of a physical imaging parameter, e.g., spatially heterogeneous speed-of-sound map, uncertain element positions and orientations, and reconstruction voxel coordinates, and optimize it based on focusing quality metrics extracted from the final reconstructed images.

In one aspect, the invention provides a method of ultrasound imaging comprising: a) performing a pulse-echo acquisition to produce channel signals using ultrasound transducers, wherein the acquisition comprises transmitting ultrasound signals using transmit elements and receiving pulse-echo responses of the transmitted ultrasound signals using receive elements; b) processing the transmitted and received channel signals to produce a dataset of a set of transmit elements and a set of receive elements; c) calculating an estimate of a critical imaging parameter; d) performing beamforming using the dataset and the estimate of the critical imaging parameter; e) calculating a desired loss function, wherein the loss function is minimized with respect to the critical imaging parameter; f) differentiating the calculated loss function with respect to the critical imaging parameter by backpropagation; g) updating the estimate of the critical imaging parameter; h) repeating steps (d)-(g) until a convergence condition is satisfied; i) generating an enhanced ultrasound image using the estimate of the critical imaging parameter.

In some implementations, the critical imaging parameter is slowness (i.e., the reciprocal of sound speed) defined at various locations and wherein the loss function is a common midpoint phase error, coherence factor, common-midpoint coherence factor or phase error of non-common midpoint sub-aperture pairs. The beamforming may comprise time-of-flight estimates computed by integrating the slowness along straight ray paths; or the beamforming may comprise time-of-flight estimates computed by integrating the slowness along bent ray paths to compensate for refraction; or the beamforming may comprise a wavefield propagator with heterogenous slowness and wavefield correlation of the transmit and received wavefield to perform beamforming.

In some implementations, the critical imaging parameter is poses (i.e., the positions and orientations) of the transmit elements and of the receive elements, and wherein the loss function is a common-midpoint phase error, phase error without common midpoint, coherence factor, common midpoint coherence factor or image entropy. The transmit elements and receive elements may be embedded in one or more flexible transducers. The channel signals may comprise multiple pulse-echo acquisitions as the transmit elements and receive elements are swept across the imaging target in any direction. The initial estimate of element poses may be obtained using motion tracking sensors, including but not limited to inertial measurement units, optical trackers, and electromagnetic sensors.

In some implementations, the critical imaging parameter is attenuation, and the loss function may be a difference between signal amplitudes.

In some implementations, the critical imaging parameter is deformation of the imaging target between pulse-echo acquisitions, comprising spatially varying translation, rotation, compression, and expansion and the loss function is the phase shift between the deformed frames. The estimate of target deformation may be used to estimate blood flow, tissue motion, or tissue elasticity.

The dataset may comprise a full synthetic aperture acquisition (i.e., the collection of all pulse-echo channel signals between each real or virtual transmit element and each real or virtual receive element).

The dataset may comprise an incomplete synthetic aperture acquisition (i.e., a subset of the full synthetic aperture acquisition), including but not limited to a transducer being swept (i.e., monostatic synthetic aperture), or a set of multiple transducers.

In some implementations an alternative, not necessarily differentiable algorithm is used to produce an initial estimate of the critical imaging parameter or to update the estimate of the critical imaging parameter in some of the iterations until convergence, including but not limited to a pretrained neural network.

In some implementations, physical quantities, element poses, and target deformation are jointly estimated and updated.

In some implementations, new channel data sample is acquired and used to update the estimated critical imaging parameter.

In some implementations, the dataset is modified by processes including but not limited to downshifting to baseband via IQ demodulation.

In some implementations, the arrangement of transmit and receive elements allows for the use of pulse-echo and/or through transmission measurements for the purpose of critical imaging parameter estimation.

In some implementations, the critical imaging parameter is embedded in the weights of a neural network where the weights act as an implicit neural representation of a physical quantity of interest.

In some implementations, the critical imaging parameter is represented using another differentiable parameterization, including but not limited to representing element poses using spline control points, where the spline coefficients may be parameterized by a neural network.

In some implementations, the loss function is regularized by physical properties and constraints of the transducers and imaging target.

In some implementations, the loss function is augmented by an auxiliary loss term that measures the discrepancy of the acquired channel signals versus simulated channel signals, in the sense of full waveform inversion, which are obtained from a differentiable ultrasound simulation that is also parameterized by unknown physical properties.

In some implementations, the transmit elements may comprise real or virtual transmit elements. The receive elements may comprise real or virtual receive elements.

In some implementations, a plurality of imaging parameters are estimated simultaneously in a volumetric imaging scenario.

### BRIEF DESCRIPTION OF THE DRAWINGS

FIG. 1 is a schematic diagram illustrating a processing pipeline for a differentiable beamforming method for ultrasound autofocusing, according to an embodiment of the invention.

FIGS. 2A, 2B illustrate common-midpoint phase error for non-corrected beamforming and corrected beamforming, respectively, according to an embodiment of the invention.

FIGS. 3A, 3B are image grids showing results of the imaging technique for a quadrant phantom and inclusion layer phantom, respectively, according to an embodiment of the invention.

FIGS. 4A, 4B, 4C are images showing results of in vivo data reconstructed with the estimated sound speed via differentiable beamforming, according to an embodiment of the invention.

### DETAILED DESCRIPTION OF THE INVENTION

Ultrasound images that are distorted by phase aberration arising from an improper imaging model can lead to inaccurate time delays in beamforming and loss of image focus. Whereas state-of-the-art correction approaches rely on simplified physical models (e.g., phase screens), the present method provides a physics-based framework called differentiable beamforming that can be used to rapidly solve a wide range of imaging problems. We demonstrate the generalizability of differentiable beamforming by optimizing the spatial sound speed distribution in a heterogeneous imaging domain to achieve ultrasound autofocusing using a variety of physical constraints based on image quality loss functions such as phase error minimization, speckle brightness, and/or coherence maximization. These loss functions are calculated on sub-aperture images which can share a common midpoint but are not required to. The method corrects for the effects of model inaccuracies in both simulation and in vivo cases by improving image focus while simultaneously providing quantitative estimates of image model parameters, e.g., sound speed distributions for tissue diagnostics. The improved imaging model leads to accuracy improvements with respect to previously published baselines. Finally, we provide a broader discussion of applications of differentiable beamforming in other ultrasound domains.

Ultrasound images are reconstructed by time sampling the reflected pressure signals measured by individual transducer elements in order to focus at specific spatial locations. The sample times are calculated to model and compensate for the time-of-flight of a wave from the elements to the desired spatial locations, often by assuming a constant speed of sound (SoS) in the medium, e.g., 1540 m/s. However, the human body is highly heterogeneous, with slower SoS in adipose layers than in fibrous and muscular tissues, and positional changes of transducer and tissue over time. If unaccounted for, these differences lead to phase aberration, geometric distortions, and loss of focus and contrast. This degradation is a fundamental limitation of current ultrasound image reconstruction and impacts downstream tasks such as diagnostics, volumetry, and registration.

Historically, phase aberration has been described using simplified phase-screen models, which assume that distortions generated from an unknown SoS can be modeled by a gross time delay offset at every element. More recently, several methods have been proposed to estimate SoS distribution of the medium from aberration measurements as a step before actual image correction. A family of these methods still relies on simplified physical models of wave propagation to derive tractable inverse problems. These include assuming a horizontally layered medium or coherent plane wavefront propagation at different angulations. To reinforce specific assumptions about SoS heterogeneity, regularization is often introduced, including total variation for focal inclusion geometries and Tikhonov regularization for smoothly varying layered SoS distributions. While these methods perform well for one class of SoS inversion problems, it is challenging to generalize their applicability to arbitrary SoS distributions, which are generally found in clinical scenarios. Work has been carried out to find more generalizable estimation based on training neural network models to end-to-end learn SoS distributions or optimize the regularization function basis. However, these methods require thousands of training instances, which can currently practically only be obtained from in-silico simulations and show challenges generalizing to real data.

In the case of flexible transducer arrays, there is substantial uncertainty in the locations of the element positions, which pose a major challenge for image reconstruction. Because the element positions are unknown, the time-of-flight to and from regions in the field of view are unknown, preventing proper focusing, much similar to the way that phase aberration degrades focusing. Furthermore, the elements may undergo rotations that affect both their transmitted pressure and receive sensitivity fields. Together, errors in the time-of-flight estimates and element sensitivity result in aberrated wavefronts after focusing, resulting in distorted point spread functions (PSFs) with poor spatial resolution. Several approaches to array shape estimation have been proposed, including the use of external physical sensors, entropy minimization, and deep learning. An iterative optimization approach has been previously proposed, where the array shape was parameterized as the sum of two sinusoids f(x)=a sin x+b sin 2x, and the “entropy” of the beam-summed image was used to estimate the coefficients a and b; this approach used TensorFlow as an automatic differentiation framework to backpropagate gradients through an algorithm resembling the standard delay-and-sum (DAS) beamformer. The challenge with this approach is that it is limited to a small set of possible or assumed geometries. In addition, no other proposed methods have accounted for the rotation of the elements in addition to the position.

Freehand 3D ultrasound imaging, which involves sweeping an ultrasound transducer by hand over the body while acquiring 1/2/3D images and then reconstructing a composite 3D volume based on the collected image sample, requires the pose of the transducer to be known along its swept path (a sequence of poses is referred to as the trajectory of the transducer). The accuracy of the pose estimation has a significant impact on the reconstructed 3D volume. The trajectory can be measured using physical devices, such as mechanical, optical, or electromagnetic sensors, or the trajectory can be estimated without sensors based on speckle decorrelation. Inertial measurement units (IMUs) have been coupled with fixtures to restrict the types of allowable motion and improve the trajectory estimation. Deep learning has also been used to incorporate IMU readings to accurately estimate the trajectory. Sensorless freehand 3D imaging is much more difficult, and trajectory estimation performance has historically been worse than sensor-enhanced imaging. Each of these approaches can be viewed as a two-stage reconstruction, where each individual 2D image is beamformed or reconstructed independently, followed by a secondary stitching of 2D images into 3D volumes, e.g., using a voxel-filling algorithm. A drawback of this two-stage approach is that trajectory estimation is performed using the envelope-detected (and often post-processed) images, and therefore does not benefit from the phase-sensitive raw ultrasound data. In other words, the image samples from multiple poses can only be used for incoherent compounding, i.e. averaging of magnitude or intensity values, and cannot be used to retrospectively create a large synthetic aperture for improved spatial resolution. In this latter case, collection of the ultrasound systems channel data during the sweep can allow for high resolution 2D and 3D images to be created via swept synthetic aperture. The accuracy of pose required for swept synthetic aperture, however, is significantly greater than that required for stitching 2D images together and have required very precise mechanical systems to measure the pose of the transducer.

The present technique extends open-source tensor libraries to model the pipeline of ultrasound image reconstruction as a composition of differentiable operations, allowing optimization based on a single data instance. Open-source tensor libraries can perform automatic differentiation of composable transformations on vector data. These libraries are the backbone of complex neural network architectures that use automatic reverse-mode differentiation (backpropagation) to iteratively optimize weights based on a set of training instances. These libraries also simplify and optimize portability to high-performance computing platforms.

We describe an ultrasound imaging technique that jointly performs imaging model property (i.e., imaging parameter) estimation and image quality enhancement via differentiable beamforming. We formulate image reconstruction as a differentiable function of the imaging model parameters, including heterogeneous sound speed, element position, medium velocity, etc. and optimize one or more based on quality metrics extracted from the final reconstructed images. For purposes of illustration, and without loss of generality, the discussion below focuses on the case where the imaging parameter is slowness (related to SoS). The estimation of other imaging parameters follows the same method.

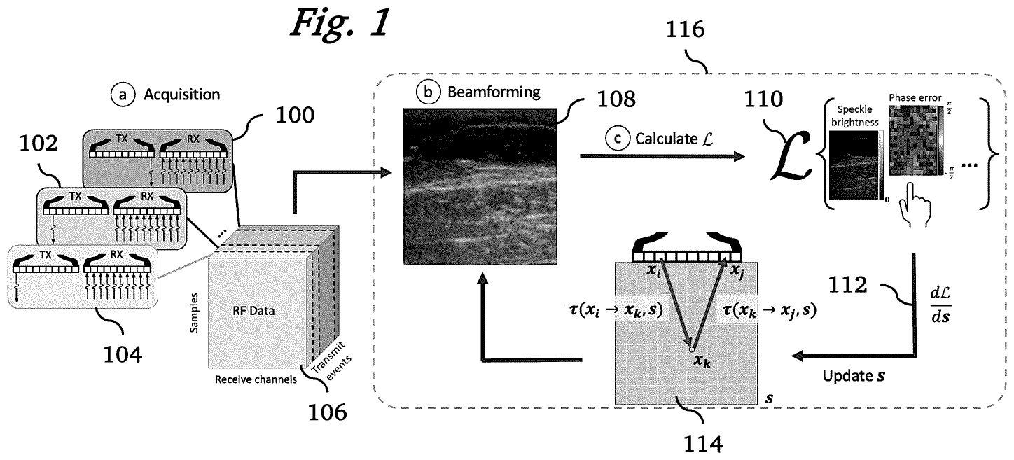

FIG. 1 shows a processing pipeline of the differentiable beamforming method for ultrasound autofocusing. In an acquisition step an initial full synthetic aperture data acquisition is performed to acquire RF channel signals 100, 102, 104. The acquisition involves transmitting ultrasound pulses using transmit elements and receiving echoes of the transmitted ultrasound pulse using receive elements. Specifically, a pulse is transmitted on one transmit element, and the echo signal is received on all other elements. This is repeated for each transmit element, such that all pairs of transmit and receive elements have acquired a signal. The transmit and receive signal pairs are typically arranged in a 3D matrix with axes of time, transmit element, and receive element. This matrix is called the complete RF data 106, but can be composed of either radiofrequency (RF), analytic (complex) data also known as in-phase and quadrature (I/Q). The data 106 is then used for straight ray integration and beamforming 108 with an initial estimate of an imaging parameter (e.g., slowness). A desired loss 110 objective function is calculated and then differentiated 112 with respect to the imaging parameter (e.g. slowness), which is then updated 114 using backpropagation and used for the next iteration of beamforming 108 using the updated imaging parameter. This sequence of steps 116 is then repeated until convergence is reached.

An ultrasound image is generated using the critical imaging parameter. The beamforming or image reconstruction process integrates or compensates for the critical imaging parameter to achieve an improved or enhanced ultrasound image. The ultrasound images in this context can be any type of ultrasound image, including B-mode, Doppler (color, power, or spectral), contrast-enhanced, etc.

As examples for each of the critical imaging parameters, in the speed of sound case, the updated estimate of the speed of sound is used to improve time of flight models used for beamforming. The output of the beamformer is then used to produce an improved ultrasound image that has compensated for the variation in speed of sound of the medium.

More generally, the critical imaging parameter can be other parameters than SoS, such as element pose, transducer pose, or attenuation. In the element pose case, the updated element pose is used to update the beamforming algorithm and produce improved ultrasound images that compensate for the unknown or uncertain positions of the transducer elements. In the transducer pose case applied to freehand 3D imaging, the series of updated transducer poses create a trajectory used to produce improved resolution in the ultrasound image/volume. In the attenuation case, the estimate of attenuation is used to apply correct for signal amplitude loss using spatial gain compensation on the ultrasound image to improve automatic homogeneity and accuracy of image brightness while spatially reconstructing a tissue parameter of attenuation.

### Beamforming Multi-Static Synthetic Aperture Data

In ultrasound imaging, the channel data represents the time series signal proportional to the pressure measured by each probe array sensor. A multi-static synthetic aperture dataset contains the channel data (or pulse-echo responses) of every pair of transmit and receive elements. We denote the signal due to the i-th transmit element, and j-th receive element as uᵢⱼ(t). This signal can be focused to an arbitrary spatial location xₖ by sampling uᵢⱼ(t) at the time corresponding to the time-of-flight τ from the transmit element at xᵢ to xₖ and back to the receive element at xⱼ, achieved via 1D interpolation of the RF signal:

$${u}_{ij} ( {x}_{k} ) = {u}_{ij} ( τ ( {x}_{i} , {x}_{k} ) + τ ( {x}_{k} , {x}_{j} ) ) . \qquad ( 1 )$$

The interpolated signals are then summed across the transmit (Nₜ) and receive (Nᵣ) apertures to obtain a focused ultrasound image:

$$u ( {x}_{k} ) = {\sum }_{j = 1 , {N}_{t}} {\sum }_{i = 1 , {N}_{r}} {u}_{ij} ( {x}_{k} ) . \qquad ( 2 )$$

This process of interpolation and summation is called delay-and-sum (DAS) beamforming.

### Differentiable Beamforming

DAS is composed of elementary differentiable operations and is consequently itself differentiable. Therefore, DAS can be incorporated into an automatic differentiation (AD) framework to allow for differentiation with respect to any desired input parameters θ. For a given loss function L(u(xₖ; θ)) that measures the “quality” of the beamforming, θ can be optimized using gradient descent to identify the optimal θ* using update steps Δθ:

$${θ}^{*} = arg {min}_{θ} L ( u ( {x}_{k} ; θ ) ) , Δ θ = θ - α \partial / \partial θ \{ L ( u ( {x}_{k} ; θ ) ) \} . \qquad ( 3 )$$

This differentiable framework is flexible, providing many ways to parameterize the beamforming. Although this approach applies to various critical imaging parameters, here we will illustrate differentiable beamforming on the task of sound speed estimation by optimizing for slowness s in a time-of-flight delay model (i.e. θ=s).

### Time of Flight Model

Here, we parameterize the slowness (i.e. the reciprocal of the sound speed) as a function of space. Specifically, we define the slowness at a set of control points as s={s(xₖ)}ₖ, which can be interpolated to obtain the slowness at arbitrary x. The time-of-flight from x₁ to x₂ is the integral of the slowness along the path:

$$τ ( {x}_{1} , {x}_{2} ; s ) = {\int }_{{x}_{1} {x}_{2}} s dx . \qquad ( 4 )$$

For simplicity and direct comparison with previous sound speed estimation models, a straight ray model of wave propagation is used.

### Loss Functions for Sound Speed Optimization

Speckle Brightness Maximization: Diffuse ultrasound scattering produces an image texture called speckle. Speckle brightness can be used as a criterion of focus quality. Written as a loss, this is the negative average pixel magnitude:

$$SB ( s ) = ( 1 / {N}_{k} ) {\sum }_{k} \vert u ( {x}_{k} ; s ) \vert = - {L}_{SB} ( s ) . \qquad ( 5 )$$

Coherence Factor Maximization: Coherence factor, defined between 0 and 1, is the measure of the coherent signal sum over the incoherent signal sum of the receive aperture. When received signals from coherent sources are in focus (i.e. in equal phase), CF achieves the maximum value of 1. For incoherent sources such as diffuse scattering in biological tissue, CF achieves a maximum values of ⅔. We use the negative CF as a loss:

$$CF ( s ) = ( 1 / {N}_{k} ) {\sum }_{k = 1 , {N}_{k}} \{ \vert {\sum }_{j} {\sum }_{i} {u}_{ij} ( {x}_{k} ; s ) \vert / {\sum }_{j} \vert {\sum }_{i} {u}_{ij} ( {x}_{k} ; s ) \vert \} = - {L}_{CF} ( s ) . \qquad ( 6 )$$

### Common Midpoint Phase-Error Minimization

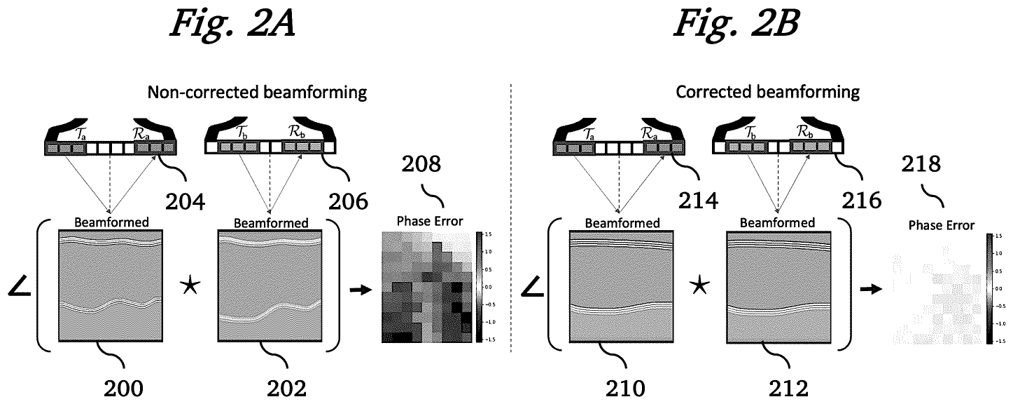

The van Cittert Zernike theorem of optics states that when imaging diffuse scatterers using a given transmit and receive sub-aperture Tₐ and Rₐ (i.e. subset of the available array elements), the resulting signal is almost perfectly correlated with the signal from a second set of apertures T_{b} and R_{b} when the two apertures share a common midpoint. The measured phase-shift between both signals should approach zero when aberration is corrected. FIGS. 2A, 2B schematically illustrate this concept of common midpoint phase error. Common midpoint phase error minimization is shown in correlated common mid-point sub-apertures. In FIG. 2A, phase error 208 for non-corrected beamforming is computed as the angle of the cross correlation of complex beamformed signals 200, 202 from different sub-apertures 204, 206 sharing a common midpoint. When the correct slowness is used for the beamforming, as shown in FIG. 2B, the common midpoint phase error is minimized. Phase error 218 for non-corrected beamforming is computed as the angle of the cross correlation of complex beamformed signals 210, 212 from different sub-apertures 214, 216 sharing a common midpoint.

We estimate the common midpoint phase shift as the complex angle between DAS signals uₐ and u_{b} of the respective sub-apertures (Tₐ, Rₐ) and (T_{b}, R_{b}), calculated using (2):

$$Δ {ϕ}_{ab} ( {x}_{k} ) = ∠ E [ {u}_{a} ( {x}_{k} ; s ) {u}_{b} * ( {x}_{k} ; s ) ] . \qquad ( 7 )$$

The common midpoint phase error (CMPE) is defined for a set of all aperture pairs (a,b) with common midpoint as

$$PE ( s ) = ( 1 / {N}_{( a , b )} ) \sum ( a , b ) \vert {Δϕ}_{a b} \vert = {L}_{PE} ( s ) . \qquad ( 8 )$$

Phase error can further be calculated on non-common midpoint sub-apertures granted the estimated phase shift due to the spatial location of the sub-aperture pair is accounted for in the error formulation.
Coherence Factor, and normalized speckle brightness, can further be calculated on common-midpoint sub-apertures to improve the coherence of the spatial coherence factor measurements.

### Implementation of a Differentiable Beamformer

A differentiable DAS beamformer was implemented in Python using JAX, which provides out-of-the-box GPU acceleration. DAS was parameterized by the slowness map, where the time-of-flights for beamforming were calculated via bilinear interpolation of the slowness along a discretized path from the transmitting element to a location of interest and from the location to a receiving element. The loss was computed on 5×5 pixel patches (λ/2 pixel spacing) on a regular 15×21 grid spanning the image. The sound speed map was then optimized via gradient descent. For the phase error loss, 17-element sub-apertures were used for beamforming. The beamformed data for every sub-aperture pair with a common midpoint were cross-correlated with a 5×5 path to compute the phase shift. We further leveraged acoustic reciprocity to combine the results for reciprocal transmit/receive sub-apertures. This phase-shift measurement was then used for the final common midpoint phase error loss. The GPU-based implementation runs in ˜300 seconds for 300 iterations on an NVIDIA RTX A6000. The code for this work can be found on GitHub.

Comparison with State-Of-The-Art Methods

As a baseline for performance comparison, the computed ultrasound tomography in echo mode (CUTE) method developed by Stahli et al. was implemented in MATLAB; this method has been shown to achieve sound speed reconstruction of both layered and focal lesion geometries. The method shows some similarities in using phase error minimization from different apertures (albeit in the angular domain) and ray tracing paths. However, it relies on a coherent plane wavefront propagation model and Tikhonov regularization to build a tractable inverse problem.

### Datasets

In silico: The CUDA-accelerated binaries of the k-Wave simulation suite were used to generate multi-static RF data of 3D phantom model acquisitions. To compare with the baseline, simulations were first generated using plane-wave transmissions (115 transmits in steering range of −28.5°:0.5°:28.5°) and then converted to FSA format using REFOCUS in the rtbf framework. A linear 128 element linear probe was simulated, with a pitch of 0.3 mm and a center frequency of 4.8 MHz with a 100% bandwidth. The simulation domain was 60×51×7.4 mm³. Iso-echoic phantoms were generated whereby the sound speed was modulated relative to the density of a region so the average brightness remained constant while the sound-speed variation introduced phase aberration.

In vivo: In vivo data was collected on a Verasonics Vantage research system with a L12-3v linear transducer (192 elements, 0.2 mm pitch, 5 MHz center frequency). Three abdominal liver views, which contained subcutaneous adipose, musculoskeletal tissue and liver parenchyma, were collected from a healthy volunteer under a protocol approved by an institutional review board.

### Results

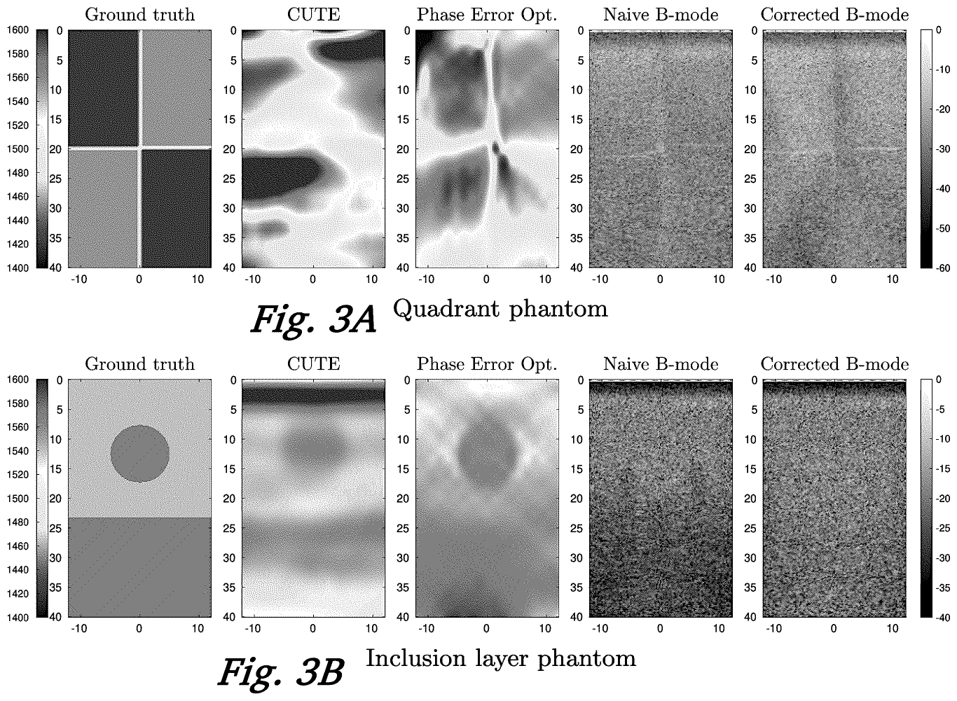

FIGS. 3A, 3B show results of the imaging technique for in silico phantom data. From left to right, each row shows the ground truth sound speed; the CUTE method as a baseline; our phase error optimization; a naive B-mode image formed assuming 1540 m/s; and the B-mode reconstructed according to our sound speed estimates. FIG. 3A shows correction of the geometric distortion at the tissue interfaces. FIG. 3B shows how the B-mode image brightness becomes more homogeneous in the lower half of the image. In the uncorrected (naive) B-modes, regions of darkening and smeared speckle can be seen as acoustic intensity diminishes due to aberration. In the quadrant phantom of FIG. 3A, a distinct spatial skewing can be observed from left to right. On the corrected images, image brightness is enhanced, iso-echogenic speckle distributions are revealed, aberrated regions are reduced, and the boundary between quadrants shows a congruent left-to-right and top-to-bottom transition. Similarly, in the inclusion phantom of FIG. 3B, characteristic triangles can be seen to the left and right of the inclusion in the naive B-mode. These triangular offshoots are artifacts produced by total wave reflection on the lateral lesion boundaries when the ultrasound wave encounters an SoS transition at grazing incidence. Moreover, diffraction of waves through the lesion lead to aberration errors behind the lesion. In the corrected B-mode, these dark regions are enhanced, and the image has an overall more homogeneous brightness pattern.

The sound speed distributions generated with differentiable beamforming are in general agreement with the ground truth sound speed distributions. For all phantoms, differential beamforming achieved lower (better) error metrics than the baseline. Homogeneous phantoms were best reconstructed via CF loss function, while inhomogeneous phantoms were best reconstructed via PE loss function.

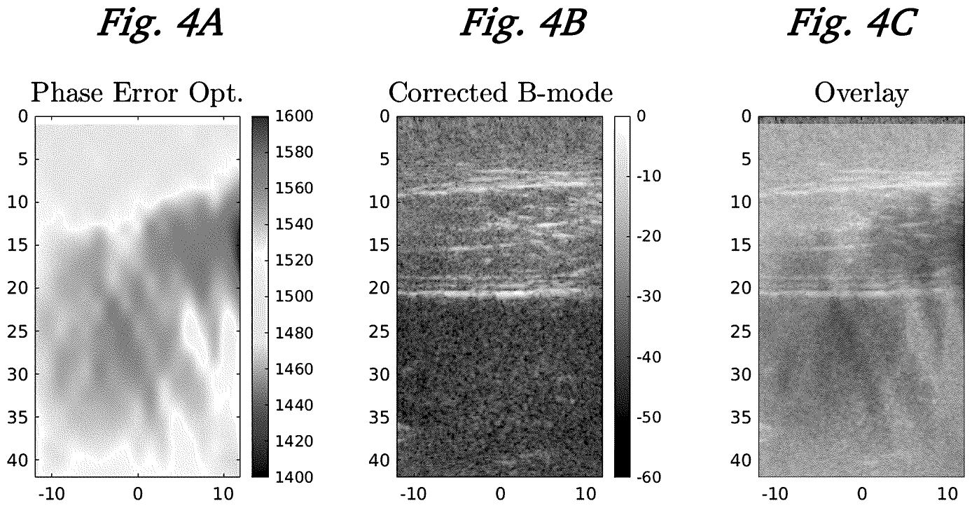

FIGS. 4A, 4B, 4C show preliminary results with differential beamforming for the in vivo data reconstructed with the estimated sound speed via differentiable beamforming. From left to right, the figure shows phase error (FIG. 4A), corrected B-mode (FIG. 4B), and the overlay image (FIG. 4C). Three layers consisting of subcutaneous adipose fat, muscle, and liver parenchyma are visible from top to bottom. The SoS reconstruction successfully delineates abdominal layers including subcutaneous adipose fat (average 1494 m/s), muscle (average 1551 m/s) and liver parenchyma (average 1530 m/s) in agreement with the literature values.

### Discussion and Conclusion

We have illustrated how differentiable beamforming can be used to solve for unknown quantities with gradient descent. Here, we parameterized beamforming as a function of the slowness and optimized with respect to several candidate loss functions, showing that common midpoint phase error was best for heterogeneous targets. The differentiable beamformer simultaneously provided B-mode image correction and quantitative sound speed characterization beyond the state-of-the-art across several challenging cases. Preliminary in vivo quantitative SoS data for liver was shown, which has direct clinical applications such as in the noninvasive assessment of non-alcoholic fatty liver disease, as well as image enhancement in general.

Importantly, the differentiable beamformer allows us to incorporate fundamental physics principles like wave propagation together with laws of wavefield imaging, reducing the number of parameters to optimize. More complex wave propagation physics, such as refraction models, can be added to SoS optimization. In addition to sound speed, this approach can be readily adapted to a broad set of applications such as beamforming with flexible arrays, where element positions are unknown, or passive cavitation mapping, where the origin of the signal is uncertain. Because the gradients flow through the entire imaging pipeline, the differentiable beamformer is also highly compatible with deep learning techniques. For instance, a model can be trained in a self-supervised fashion to identify optimal sound speed updates to accelerate convergence. Differentiable beamforming also enables the end-to-end optimization of imaging parameters for downstream tasks in computer-aided medical diagnostics.

The method is not limited to estimating SoS imaging parameter or the proposed common-midpoint phase error. It can also be used to estimate of the poses (i.e., the positions and orientations) of the transmit elements and of the receive elements, where differentiating the calculated loss function is performed with respect to element poses with suitable loss functions and backpropagation. The method can be used to estimate of the deformation of the imaging target between pulse-echo acquisitions. These deformations may include spatially varying translation, rotation, compression, and expansion. In this case, differentiating the calculated loss function is performed with respect to target deformation by backpropagation.

### Flexible Array Shape Estimation

Here we provide details of a further embodiment of the invention that performs end-to-end physics-based differentiable beamforming (DBF) together with a phase error loss to estimate the shape of the array. We use a fully differentiable implementation of DAS beamforming that accounts for both the position and orientation of each transmit and receive element.

A generic DAS beamformer was developed for arbitrary transmit sequences. This beamformer is capable of handling focused, diverging, or plane wave transmit sequences (where each transmit focus is treated as a virtual element) or any arbitrary transmit sequence by “refocusing” to retrieve the multi-static (i.e. single-element transmit) data. In all cases, the transmit sequence was used to form a synthetic transmit aperture. Additionally, the beamformer was designed to account for element directivity via an “acceptance cone” defined in the field of view.

Consider a real or virtual transmit element and a real or virtual receive element with arbitrary positions and orientations (xₜ, θₜ) and (xᵣ, θᵣ), respectively. Assuming a constant sound speed c₀, the roundtrip time-of-flight to some arbitrary voxel xᵥ∈R³ is computed as:

$$τ ( {x}_{v} , {x}_{t} , {x}_{r} ) = ‖ {x}_{v} , - {x}_{t} , ‖ + ‖ {x}_{v} , - {x}_{r} , ‖ .$$

In our generic DAS implementation, the echo at voxel xᵥ for transmit t and receive r is reconstructed by sampling the original pulse-echo data Dₜᵣ at time τ(xᵥ, xₜ, xᵣ), e.g., via linear or cubic interpolation; the echo is further weighted by transmit and receive acceptance cones to model element directivity and to restrict contributions to the field of view in front of the element. Let

$$T = {\{ ( {x}_{t} , {θ}_{t} ) \}}_{t = 1 , {N}_{t}}$$

$$R = {\{ ( {x}_{r} , {θ}_{r} ) \}}_{r = 1 , {N}_{r}}$$

denote the transmit and receive apertures, respectively, and let D be the total pulse-echo dataset. Then, the DAS beamformed signal at xᵥ due to dataset D obtained with apertures T and R is:

$$u ( {x}_{v} , D , T , R ) = {\sum }_{t = 1 , {N}_{t}} {\sum }_{r = 1 , {N}_{r}} {D}_{t r} ( τ ( {x}_{v} , {x}_{t} , {x}_{r} ) ) \times exp ( j 2 π {f}_{demod} τ ( {x}_{v} , {x}_{t} , {x}_{r} ) ) \times {h}_{cone} ( {x}_{v} - {x}_{t} , {θ}_{t} ) {h}_{cone} ( {x}_{v} - {x}_{r} , {θ}_{r} ) ,$$

where the complex exponential is used for phase compensation for demodulated baseband signals. Because each of the individual operations is differentiable, the total beamforming operation is also differentiable. Finally, the magnitude of the complex beamformed signal (i.e. the detected envelope) is referred to as the B-mode image.

From the above general equation, any of the parameters (or shift in parameters) of the above equation can be estimated. For example, to estimate tissue deformation, the shift in the parameter xᵥ is estimated. This is usually done by defining the parameter of interest as a function of the loss function.

Generally, adaptation of the differentiable beamformer framework to estimate imaging parameters is as follows:
(1) Adapt the beamforming/model equation to include the parameter to be estimated (if it is not already in the above beamforming model)
(2) Select a loss function that, when minimized, yields the optimal parameter values for the beamforming model.
(3) Parameterize the loss function by the parameter to be estimated

As an example of another embodiment of the invention, the differentiable beamformer adapted to attenuation estimation uses the same beamforming model as described above, but is updated to include an attenuation term: u_{α}(xᵥ, D, T, R, α)=u(xᵥ, D, T, R)Πᵢ₌₁^{N}^{ᵥ} exp(−αmᵢlᵢ). Here, α is the attenuation coefficient and mᵢ and lᵢ are the midpoints and lengths of each segment along the ray-path of the signal u(xᵥ, D, T, R).

A loss function is created that goes to zero when the attenuation in the above model is correct. The loss function here is shown to be a function of the attenuation (α). In this case, the loss term is a simple difference of the logs of two signals: £(α)=∥ln u_{α,1}(xᵥ, D, T, R, α)−ln u_{α,2}(xᵥ, D, T, R, α)∥, where the subscripts 1 and 2 represent the two signals, which could be two common midpoint signals from the same medium (i.e. the reference-less model) or from different mediums (i.e. reference phantom model) where one medium is the medium of interest and one is from a reference phantom of known attenuation.

1. A method of ultrasound imaging comprising:
a) performing a pulse-echo acquisition to produce channel signals using ultrasound transducers, wherein the acquisition comprises transmitting ultrasound signals using transmit elements and receiving pulse-echo responses of the transmitted ultrasound signals using receive elements;
b) processing the transmitted and received channel signals to produce a dataset of a set of transmit elements and a set of receive elements;
c) calculating an estimate of a critical imaging parameter;
d) performing beamforming using the dataset and the estimate of the critical imaging parameter;
e) calculating a desired loss function, wherein the loss function is minimized with respect to the critical imaging parameter;
f) differentiating the calculated loss function with respect to the critical imaging parameter by backpropagation;
g) updating the estimate of the critical imaging parameter;
h) repeating steps (d)-(g) until a convergence condition is satisfied;
i) generating an enhanced ultrasound image using the estimate of the critical imaging parameter.

2. The method of claim 1 wherein the critical imaging parameter is slowness (i.e., the reciprocal of sound speed) defined at various locations and wherein the loss function is a common midpoint phase error, coherence factor, common-midpoint coherence factor or phase error of non-common midpoint sub-aperture pairs.

3. The method of ultrasound imaging as in claim 2, wherein the beamforming comprises time-of-flight estimates computed by integrating the slowness along straight ray paths.

4. The method of ultrasound imaging as in claim 2, wherein the beamforming comprises time-of-flight estimates computed by integrating the slowness along bent ray paths to compensate for refraction.

5. The method of ultrasound imaging in claim 2 wherein the beamforming comprises a wavefield propagator with heterogenous slowness and wavefield correlation of the transmit and received wavefield to perform beamforming.

6. The method of claim 1 wherein the critical imaging parameter is poses of the transmit elements and of the receive elements, and wherein the loss function is a common-midpoint phase error, phase error without common midpoint, coherence factor, common midpoint coherence factor, or image entropy.

7. The method of ultrasound imaging as in claim 6, wherein the transmit elements and receive elements are embedded in one or more flexible transducers.

8. The method of ultrasound imaging as in claim 6, wherein the channel signals comprise multiple pulse-echo acquisitions as the transmit elements and receive elements are swept across the imaging target in any direction.

9. The method of ultrasound imaging as in claim 6, wherein the initial estimate of element poses is obtained using motion tracking sensors.

10. The method of claim 1 wherein the critical imaging parameter is attenuation, and wherein the loss function is a difference between signal amplitudes.

11. The method of claim 1 wherein the critical imaging parameter is deformation of the imaging target between pulse-echo acquisitions, comprising spatially varying translation, rotation, compression, and expansion; and wherein the loss function is a phase shift between deformed frames.

12. The method of ultrasound imaging as in claim 1, wherein the dataset comprises a full synthetic aperture acquisition.

13. The method of ultrasound imaging as in claim 1, wherein the dataset comprises an incomplete synthetic aperture acquisition.

14. The method of ultrasound imaging as in claim 1, wherein a pretrained neural network is used to produce an initial estimate of the critical imaging parameter and to update the estimate of the critical imaging parameter in at least one iteration.

15. The method of ultrasound imaging as in claim 1, physical quantities, element poses, and target deformation are jointly estimated and updated.

16. The method of ultrasound imaging as in claim 1, wherein a new channel data sample is acquired and used to update the estimated critical imaging parameter.

17. The method of ultrasound imaging as in claim 1, wherein the dataset is modified by downshifting to baseband via IQ demodulation.

18. The method of ultrasound imaging as in claim 1, wherein the critical imaging parameter is embedded in the weights of a neural network where the weights act as an implicit neural representation of a physical quantity of interest.

19. The method of ultrasound imaging as in claim 1, wherein the critical imaging parameter is represented using spline control points to represent element poses, wherein the spline coefficients are parameterized by a neural network.

20. The method of ultrasound imaging as in claim 1, wherein the loss function is regularized by physical properties and constraints of transducers and an imaging target.

21. The method of ultrasound imaging as in claim 1, wherein the loss function is augmented by an auxiliary loss term that measures a discrepancy of acquired channel signals versus simulated channel signals.
